(19) United States
(12) Patent Application Publication
(10) Pub. No.: US 2004/0180006 A1
(43) Pub. Date: Sep. 16, 2004

(54) Methods and compositions for blocking the calcium cascade

(76) Inventor: Nicholas A. Sceusa, New York, NY (US)

Correspondence Address: BROWDY AND NEIMARK, P.L.L.C., 624 NINTH STREET, NW, SUITE 300, WASHINGTON, DC, 20001-5303, US

(21) Appl. No.: 10/734,155

(22) Filed: Dec. 15, 2003

Related U.S. Application Data
(60) Provisional application No. 60/453,833, filed on Mar. 12, 2003.

(51) Int. Cl. A61K 33/34; A61K 33/32; A61K 33/26; A61K 33/06; A61L 9/04; A61K 9/14

(52) U.S. Cl. 424/46; 424/630; 424/641; 424/639; 424/646; 424/682

(57) ABSTRACT

Methods and compositions are provided for blocking the calcium cascade. More particularly, compositions and methods are provided that inhibit histamine reactions triggered by the calcium cascade, using the naturally occurring pH gradients in the body to deliver therapeutic doses of metal ions which block the calcium cascade.

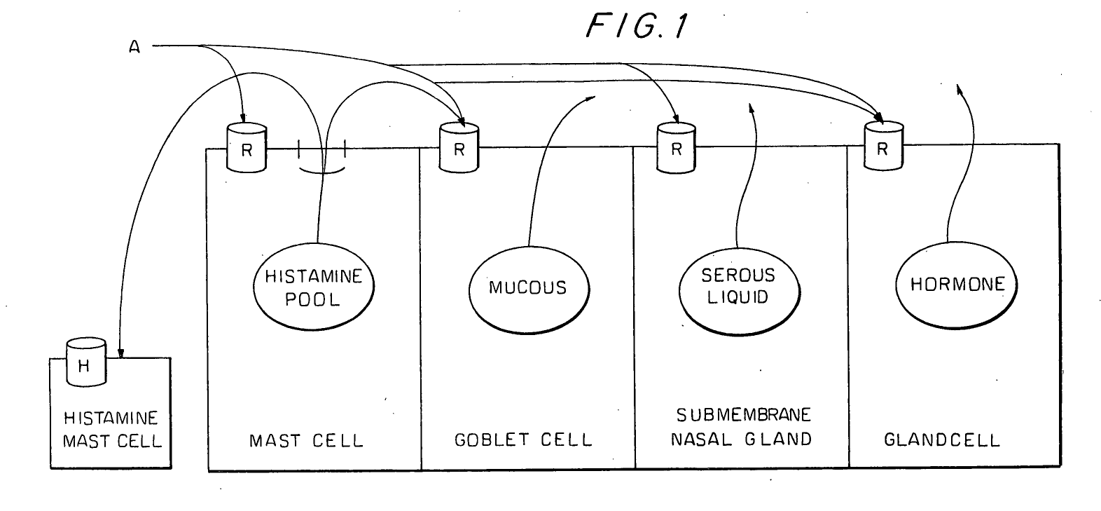

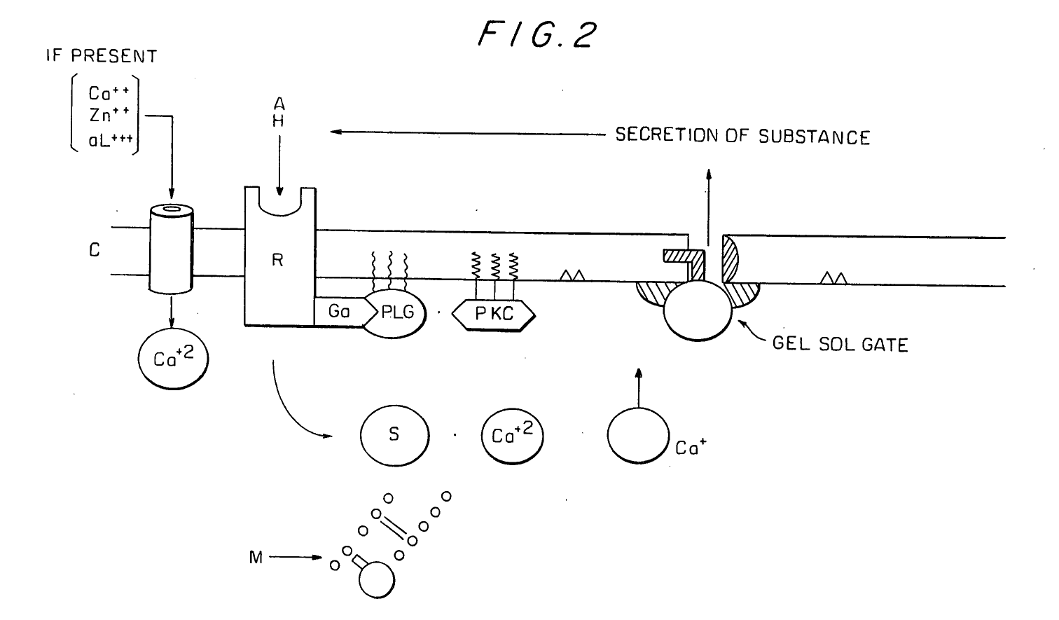

### CROSS-REFERENCE TO RELATED APPLICATIONS

This application claims priority from provisional application No. 60/453,833, filed Mar. 12, 2003, the entire contents of which are hereby incorporated by reference.

### FIELD OF THE INVENTION

The present invention relates to methods and compositions for blocking the calcium cascade. More particularly, the present invention relates to compositions and methods that inhibit histamine reactions triggered by the calcium cascade, using the naturally occurring pH gradients in the body to deliver therapeutic doses of compounds which block the calcium cascade.

### BACKGROUND OF THE INVENTION

Common colds, which are acute viral infections of the nose normally caused by rhinoviruses, are the most common acute illness in the United States and account for nearly one half of all lost school days and lost work days. Rhinitis is another term for such an inflammatory disorder of the nasal passages. The symptoms of rhinitis typically consist of sneezing, rhinorrhea, nasal congestion, and increased nasal secretions. Failure to treat rhinitis may lead to other disorders, including infection of the sinuses, ears, and lower respiratory tract.

Two types of oral medication are typically used to treat rhinitis: decongestants and antihistamines. Decongestants and antihistamines differ in mechanism of action, therapeutic effects, and side effects. Many rhinitis formulations combine both decongestants and antihistamines.

Decongestants act to constrict vessels in the nasal mucous membranes and thereby decrease tissue swelling and nasal congestion. Like adrenaline, nasal decongestants are stimulatory and produce side effects which may be tolerated during the day. However, decongestants may produce nervousness, restlessness, and insomnia if taken at night, which inhibits sleep.

Histamine is a mediator released from cells which line the walls of the nasal mucous membranes (mast cells). When released, histamine is known to bind to local receptors and thereby cause sneezing, nasal itching, swelling of the nasal membranes, and increased nasal secretions. Antihistamines relieve these effects, albeit by a different mechanism than decongestants. Antihistamines block the binding of histamine to histamine receptors in the nasal membranes. Antihistamines preemptively bind to histamine receptors and are effective only if given prior to histamine release (once histamine is released and binds to the receptor, it is too late). Although individuals typically take antihistamines after symptoms occur, it is more desirable to dose antihistamines so as to effect therapeutic activity in anticipation of the peak times of histamine release.

Older antihistamines are sedating, although newer antihistamines with little or no sedative properties have been developed.

It is well known that individuals with rhinitis use antihistamines and decongestants hundreds of millions of time a year. It is not uncommon for inappropriate choices of antihistamines and/or decongestants to result in symptomatic worsening rather than improvement.

In addition to antihistamines and decongestants, colds have been treated with soluble and ionizable zinc compounds applied to the oral and oralpharyngeal mucosa. Detailed study of zinc compounds used to treat common colds has revealed that certain zinc compounds were more effective in treating colds than other zinc compounds, and some other compounds were completely ineffective. The basic conclusion derived from these findings relates to the presentation and availability of positively charged zinc ions for rapid antirhinoviral, antihistamine-like effects, as zinc ion is widely reported to have these properties in vitro, if not necessarily in vivo.

It has been determined that to be effective in treating colds, the zinc must be present as positively charged ions to produce the most rapid results, see Eby, U.S. Pat. No. 5,286,748. In this patent Eby describes treating colds by holding a composition containing an active ingredient in the mouth for from one to three hours in order to bring about the reduction in duration of common colds or they symptoms. Eby thus teaches that the efficacious use of antiviral agents, antirhinoviral agents, interferons, interferon inducers, T-cell mitogens, decongestants, drying agents, astringents, antihistamines, protein precipitators, antibradykinins, and all other pharmaceutical agents for treating common colds is enhanced and optimized when these medicaments are administered through the oral and oral pharyngeal tissue route of administration by means of lozenges or similar solid or liquid means of administering these medications.

While previous workers have administered antihistamines and decongestants to treat the symptoms of rhinitis, there has been no way to inhibit production of histamines per se so as to prevent the adverse effects resulting from production of histamines.

### SUMMARY OF THE INVENTION

It is an object of the present invention to overcome the aforesaid deficiencies in the prior art.

It is another object of the present invention to provide methods and compositions for administering pharmaceutically effective amounts of therapeutic ions that block or inhibit the calcium ion cascade.

It is a further object of the present invention to provide methods and compositions for administering pharmaceutically effective amounts of therapeutic ions that block the calcium ion cascade and halt the histamine reactions triggered thereby.

It is still another object of the present invention to use the body's naturally occurring concentration or pH gradients to deliver therapeutic doses of calcium blocking ions.

The present invention provides a method and composition for formulating dosage forms of pharmaceutically effective amounts of therapeutic metal ions to be delivered from a repository body compartment or organ (e.g., the mouth) to a recipient body compartment or organ (e.g., the nasopharyngeal area) based upon the Teorell Meyer gradient of differing pH levels between these two compartments.

It is yet another object of the present invention to inhibit autoimmune component diseases which cause secretions and eruptions via the calcium cascade.

The general means for administration of biologically active agents using the Teorell Meyer gradients is disclosed in Sceusa, U.S. Pat. No. 6,414,033, the entire contents of which are hereby incorporated by reference.

In the present invention, this method of administration is used to administer pharmaceutically effective metal ions and combinations of ions to swamp the calcium ions naturally occurring in the body so as to block the calcium cascade, thereby inhibiting the formation of histamine.

### DETAILED DESCRIPTION OF THE INVENTION

The method of the present invention can be used to treat immune and autoimmune component diseases and conditions which cause secretions and eruptions via the calcium cascade. Included in this treatment are rhinitis, rashes, hives, blistering eruptions running sores such as the bullous form of impetigo, and cold sores.

Calcium ion, Ca²⁺, is a ubiquitous intracellular regulator of cellular function. Levels of Ca²⁺ are controlled by a variety of channels, exchangers, and pumps found in the plasmalemma and internal membranes. Cell stimulation causes the intracellular Ca²⁺ to increase transiently. This second-messenger signal is then mediated by Ca²⁺-binding proteins. There are three primary molecular mechanisms for transmitting the signal: calmodulin binds to specific sites on target proteins and activates enzymes by depressing the active site. In the presence of Ca²⁺, the annexins have a strong affinity for phospholipids. Annexins can form an interlocking network along membrane surfaces and alter membrane fluidity. Annexins are important in regulating membrane ion conductance. Protein kinase C is regulated by Ca²⁺, phospholipid, and diacylglycerol. Cellular studies using specific activators and inhibitors of protein kinase C have shown this Ca²+ pathway to be involved in cell growth, differentiation, and development of tumors.

In many cells, Ca²+ is the key signal for triggering exocytosis.

Electrical depolarization, K+ depolarization, and nicotine stimulation open the Ca²⁺ channels, allowing an immediate influx of Ca²⁺ ions from the external medium. The increase in [Ca²⁺] is restricted to the immediate vicinity of the plasma membrane after activation of Ca²⁺ channels. Recent experimental evidence confirms that when Ca²⁺ channels are activated, the concentration of Ca²⁺ at the secretory sites could range from 10 to 100 μM, creating a Ca²+ microdomain.

Mast cells are able to synthesize, store, and secrete histamine. However, Ca²⁺ alone is not able to trigger histamine secretion, but requires a guanine nucleotide together with Ca²⁺ for exocytosis.

Mast cells are stimulated by antigenic binding on cell surface IgE receptors or by compound 48/80. A sudden rise in Ca²⁺ concentration [released from an InsP3 (inositol 1,4,5-triphosphate) -sensitive pool] is observed, reaching a level of up to several micromolar. The signal is transient and [Ca²⁺] concentration declines within several seconds to its original baseline value. Degranulation begins soon after this transient response.

Two important features should be noted concerning [Ca²⁺] and secretion in mast cells:

a. the [Ca²⁺] transient is not dependent on the presence of Ca²⁺ in the external medium; and

b. simultaneous recording of capacitance and membrane conductance reveals that conductance is constant throughout the duration of exocytosis.

These results indicate that Ca²⁺ entry through Ca²⁺ channels is not a required signal for exocytosis, but rather its release from internal stores. A Ca²⁺ current, called ICRAC (calcium-release activated calcium) was described in mast cells (Hoth and Penner, 1992). ICRAC is activated by the depletion of intracellular Ca²⁺ ions. The role of ICRAC in nonexcitable cells could be to maintain a high [Ca²⁺] and to replenish empty Ca²⁺ stores after stimulation.

One conclusion is that Ca²⁺ release from internal stores is sufficient for exocytosis in mast cells. However, intracellular application of InsP3 induces a transient [Ca²⁺] increase but does not trigger secretion, emphasizing that the signal is more complex. Application of the nonhydrolyzable GTP analog GTP-γS (100 μM) inside a mast cell (via a patch pipette) induces a transient [Ca²⁺] increase, after a delay of 10-20 seconds, followed by an increased in capacitance. GTP-γS activates G protein, including the Gq protein coupled to PLC, with a resulting production of InsP3 and DAG. Ca²⁺ is released from the InsP3-sensitive pool by the same mechanisms as with the direct application of InsP3 described above. However, in this case, exocytosis is stimulated, leading to the conclusion that GTP-γ-S activates a G protein that plays a crucial role in exocytosis. This part of the mechanism does not affect the administration of extraneous ions.

As discussed above, Ca²⁺ and a guanine nucleotide are necessary and sufficient effectors to ensure secretion in mast cells. The stable analog GTP-γS is the commonly used guanine nucleotide, although any ligand that binds to G protein is able to stimulate secretion. In chromaffin cells, two types of observations have been recorded:

a. GTP-γS increases the Ca²⁺ sensitivity of secretion in permeabilized cells; and

b. GTP analogs stimulate secretion in a Ca²⁺-independent manner. GTP analogs do not enhance the secretion induced by high Ca²⁺ concentration (10 μM), which indicates that the two stimuli, GTP and Ca²⁺ act on the same exocytotic pathway.

The role of the sodium pump in the plasma membrane potential changes induced by compound 48/80, a proprietary compound that acts to elicit or provoke release of mast cells, and by antigenic challenge has been investigated using a fluorescent potential sensitive probe, bis-oxonol. Compound 48/80 induced a fast decrease of the fluorescence of bis-oxonol followed by a delayed decrease. The antigenic stimulation induced only a delayed decrease of fluorescence. Zinc gluconate inhibited the first decrease but did not alter the second one. The delayed decrease was inhibited by ouabain or by the absence of potassium. These results suggest that compound 48/80 induced mast cell secretion via a zinc-sensitive mechanism followed by activation of the sodium pump. The changes in the plasma membrane potential during the antigenic stimulation are due to the activation of the sodium pump but occur after the secretion process. (Agents Actions 1991, 33(1-2):88-91).

The effects of zinc gluconate have been studied on rat peritoneal mast cells and rat basophilic leukemia cells (RBL 2H3) stimulated by various secretagogues. The IC50′2 of zinc gluconate on peritoneal cells were (μM):1.6, 1.9, 5.4 and 18 for ionophore A23187, phorbol 12-myristate 13-acetate, substance P, and imunoglobulin E-antigen, respectively. Higher concentrations of zinc gluconate were required to inhibit histamine secretion from RBL 2H3 cells, i.e., !.2 μM (ionophore A23187) and 140 μM (immunoglobulin E-antigen). Zinc gluconate (10-4 to 10-3 M) also inhibited the IgE-dependent contraction of guinea pig trachea, but was unable to affect that induced by endogenous histamine. These results suggest that zinc Gluconate acts intracellularly and is selective of “typical” or “connective tissue” mast cells. This effect holds true for all divalent metal ions that are hard acids, i.e. those not in the Calcium column of the periodic table. Zinc and copper are both physiological, but aluminum and tin will also work.

In a preferred embodiment of the present invention, therapeutic doses of ions are used to block the production of histamine triggered by the calcium ion cascade. The calcium ion current (ICRAC, calcium release activated calcium) present in mast cells is activated by the depletion of intracellular calcium ion pools. The function of this calcium ion current is presumably to maintain a high calcium ion concentration in n. Interfering with this calcium ion current, then, would affect the ability of mast cells to replenish calcium stores, halting the cascade.

According to the present invention, administering ions that interfere with the calcium cascade, such as pharmaceutically acceptable polyvalent ions, will block the calcium cascade by swamping it. These polyvalent ions take the place of the calcium ion, so that the cascade is interrupted. Calcium acts as a trigger for the kinases that produce the mucous from the goblet cells and sub-mucosal glands associated with rhinitis and thus produces probable positive charges in mucociliary clearance. The other ions block the kinases by attaching to the kinases but not triggering them. These ions also interfere with mast cell release of histamine, which is associated with allergic reactions rather than the common cold.

Among the polyvalent ions that can be used in the present invention are Zn²⁺, Cu²⁺, Al³⁺, Fe³⁺, Sn²⁺ and Mn²⁺, either alone or in combination with each other. Calcium has a large diffuse electron cloud, and the other ions have smaller, tighter electron clouds, so they slip into the spots in the calcium cascade that calcium occupies, and then displace calcium by the law of mass action. Since the ions are correctly charged, they remain in the nasopharyngeal area after the dosage form is consumed. The polyvalent ions that can be used in the present invention can be any positively charged polyvalent ions that fit the receptor site. Organic cations or organo-metallic cations that fit the receptor site can be used as well to block the calcium cascade.

Teorell-Meyer dosage forms depend upon bioelectricity for their function. A biologically closed electric circuit (BCEC) is physiologically analogous to an ordinary electric circuit, except that ions, predominantly, as well as electrons, move along and through the circuit. In biological material, the co-transport of electrons occurs in short redox steps. Ions are transported electro-osmotically. Concentration, and consequently, electrical gradients, are maintained by Donnan Equilibria, which are large sheets of charge in the tissue proteins, and by ion pumps functioning at the expense of ATP. The second half of the circuit, the return half, takes place via passive or facilitated diffusion. Ions will follow, or respond to the flow of current according to their net charge, from one are of charge density to another area of different charge density, as part of the usual BCEC circulation. The local viscosity, and the electrical path length, which is a vector quantity, plays an important role. Vectors have the properties of force, distance (length), according to the gradients that compose them. Controlling the electrical vector makes it possible to control the ion, because the electrical vector is very many times stronger than any of the other which act.

Although a BCEC is electrically closed, it is thermodynamically and physiologically open, which makes it possible to place a dosage form in a predetermined location. This property is used to artificially induce a gradient, using appropriate buffering, companion, and carrier molecules. Certain molecules may act as all three at the same time, and the amino acids and their congeners are ideal for this purpose. By introducing the specially designed and buffered dosage form, the pH of the recipient compartment, in which the form is placed, is changed relative to the target compartment, setting up the induced gradient and corresponding concentration cell. This is provided for by the Lewis acid-base definition, which considers all positive charges as acids and all negative charges as bases.

Inducing the pH change and controlling the bioelectrical field and corresponding electrical vector makes it possible to manipulate the direction of ionic flow and transport. Since the electrical vector is many times more powerful than the other vectors acting, the ionic flow can be stopped or reversed for the time the induced field is present. If the electrical vector is coupled to act in the same direction as the other vectors, the effect is most powerful. The three vectors which are known to act are the hydrostatic vector, the particulate (colligative) vector, and the electro-motive force (electro-osmotic) vector.

It should be remembered that the association constant (Ka) and its reciprocal, the dissolution constant, Kd, for any complex are pH dependent. In the context of an electrical gradient inside a concentration cell, these constants may also be considered to be electrically dependent. In other words, at one pH a complex may be completely associated, and at another pH, almost completely dissociated.

Therefore, for any given complex, the concentration cell has a continually changing spectrum of pH and association constants inherent within it. This change over distance, which operates most strongly at the endpoints, is what allows the system to deliver ions in the way it does.

Charged particles do not easily penetrate membranes, because charged particles are generally not lipid soluble. This is generally true, but is not universal. If a particle is fairly small and its charge comparatively large, and the membrane relatively thin, an ion will be dragged through the lipid bi-layer membrane. By arranging the electrical vector in the same direction as the other diffusion vector, this penetration can be greatly improved. This is particularly useful for ions delivered perpendicular to the membranes, for example, the thin membranes of the nasal conchae in the nose.

The mouth-nose circuit has a natural partition in the hard and soft palates, which can be easily modeled as an electrophoretic sheet. The fluids of the nasal cavity are continually oxidized by breathing, while the oral cavity is usually closed, except for speech or exhalation. Expression of CO₂ during speech or exhalation forms the basic bicarbonate ion (HCO₃⁻) in the saliva. These natural processes maintain the two compartments in different states of oxidation, with the nose at a lower pH than the mouth. This gradient is maintained homeostatically, and results in a concentration cell. This concentration cell can be easily observed using a oscilloscope or sensitive volt meter. Currents between these two compartments can be measured in the neighborhood of 80-120 millivolts. These can be detected by touching a probe or a wick electrode to the mucosa of both compartments.

Based upon the relative difference in size between the blood compartment and that of the liquid and mucosa in the nasal cavity and sinuses, it is estimated using a spherical approximation that a difference of at least three orders of magnitude exists between these two compartment, based upon their volumes. If a delivered dose is divided equally between them, one can readily see how the resulting concentration differences make it possible to achieve pharmacologically active concentrations in the nose while avoiding systemic side effects.

The ions designed to block the calcium cascade are administered to the nasopharyngeal area by transport across the mucous membranes of the mouth by virtue of the Teorell Meyer gradient. In the mouth-nose compartment system, fluids, saliva an other liquid and semi-solid substances are directed by the natural action of teeth, tongue and saliva flow in the direction of the digestive system, unless forcibly expelled. However, according to the present invention, pH, electrical, and other forces combine in causing reversal of the natural flow of ions from nose to mouth. The contribution made by the electrical potential force is evaluated using the Boltzmann equation as follows:

$$\begin{matrix}d ϕ = 2.303 kT / zq log {K}_{eq} \\ and & ( 5 ) \\ Em = \frac{- 61.5 \times {10}^{- 3}}{z} [ log {K}_{eq} ] & ( 6 )\end{matrix}$$

where z=valence of the ion. Since K_{eq}=[products]/[reactants]={oxidized}/{reduced}, by substitution

$$Em = \frac{- 0.0615 volts}{z} log \{ [ oxidized ] / [ reduced ] \} \qquad ( 7 )$$

Since z for H⁺ is one (1), we may calculate, using the average figures in Table 1, using the Boltzmann equation:

Enose to mouth=−0.0615×log{[10⁻⁶[/]10^{-7.2}7.2]} (8)

Enose to mouth=−7.812×10⁻² volts ( 9)

Emouth to nose=+7.812×10⁻² volts ( 10)

This result may be substituted into the flux equation above, to calculate the actual movement of ions. To examine the flux of (H⁺] in the naso-pharaynx, we can calculate as follows:

$$\begin{matrix}{J}_{H +} = D \frac{- 2.6 log [ Nose ] / [ Mouth ]}{dx} \{ {C}_{nose} - {{C}_{mouthC}}^{- 2.6 log [ Nose / Mouth ]} \\ or & ( 11 ) \\ {J}_{H +} = 3.208 \times {10}^{- 8} mole cm \text{/} l sec & ( 12 )\end{matrix}$$

The ions are administered in the form of their non-toxic salts which have a pH such that the ions can be delivered from the mouth to the nose. Since the tissues of the nose and mouth are either positively or negatively charged, the metal salt is chosen to reach the nose. Carrier/facilitator molecules, such as the smaller amino acids, aldehydes, sugars, amino sugars, and related polysaccharide and other polymeric compounds can be used to help buffer, help change pH conveniently, and help propel the ion to its site of action.

Thus, to move a positively charged ion, such as the metal ions used in the present invention, from the mouth to the nose, the mouth pH must be lowered below the target or destination area for the ion. The movement is osmo-electrophoretic, and the energy for carrying the ion from the mouth to the nose is supplied by the Teorell-Meyer concentration gradient between the nose and mouth.

To move the desired ions from the mouth to the nose, the metal salts are formulated to include ingredients which are buffers or buffer pairs or which are capable of maintaining the pH of the formulation and the mouth at the desired level. Buffers may be single compounds such as solutions of amino acids, Trigg, and other compounds containing both acid and basic groups on the same molecule. Buffers usually are systems containing two components, namely, a salt and its correlative acid or base. However, a buffering system may be complex, containing several components. A multiple component buffer system is a system containing several acids and their correlative salts. It may also contain non-related salts or amino acids or similar zwitterionic compounds. These acids and salts are usually organic, but they may be inorganic.

An example of an inorganic physiological buffer is the phosphate buffer system comprising phosphate buffer pairs (sodium, potassium, or other ligands) having the chemical forms L₂HPO₄, LH₂PO₄. Carbonate buffers generally have the chemical form LHCO₃, where L is a suitable monovalent ion. Zwitterionic compounds can be used, such as all twenty GRAS listed amino acids. Also, GRAS listed acids, aldehydes, sugars, carbohydrates, substituted carbohydrates, or other compounds, alone or in combination, which may be used as buffers or as buffer pairs. Artificial buffer systems have also been commercially developed for pharmaceutical use, and are suitable for use in the present invention.

A preferred form of ions to be administered is a combination of zinc ion and copper 2+ ion in a ration of from about 2:1 to about 1:2 by equivalents. This is the approximate ratio of copper and zinc naturally occurring in the blood: copper is present at about 11-22 micromoles per liter of blood, and zinc is present in an amount of about 11.5-18.5 micromoles per liter of blood. Examples of salts to be used include glycinate and acetate salts of zinc and/or copper. This combination of copper and zinc involves no induced ion imbalance. Since copper is a smaller ion than zinc, it is delivered to the recipient compartment more rapidly, allowing for a rapid onset of attack on the enzyme cascade.

The ions used in the present invention can be in any pharmaceutically acceptable form as salts. A nonlimiting list of salts that can be used to block the calcium cascade according to the present invention are copper gluconate, copper sulfate, ferrous sulfate, ferrous fumarate, ferrous gluconate, ion carbonyl, magnesium oxide, magnesium carbonate, magnesium hydroxide, magnesium gluconate, magnesium aspartate, magnesium orotate, magnesium oxide, magnesium hydroxide, manganese sulfate, manganese gluconate, zinc sulfate, zinc acetate, zinc gluconate, zinc citrate, zinc dipicolinate, zinc aspartate, zinc orotate, and amino acid chelates of any of the above metal ions, alone or in combination with other metal ions.

All anions which can mildly chelate the metal ion to be administered in such a way that release of the ion in the recipient compartment is easy (high Kd) can be used. The preferred ions for use in the present invention are physiological divalent ions which are not in the calcium period of the periodic table, including zinc, copper, manganese, magnesium, and aluminum. Of the transition metals, the preferred metals are manganese, iron, copper, cobalt, and zinc. To minimize toxicity, physiological ions are preferred.

The metal ions can be formulated in any convenient dosage with carries, excipients, buffers, etc. which are pharmaceutically acceptable. Pharmaceutically acceptable carriers, such as fructose, sucrose, dextrose, maltose, lactose, sweetened water and the like, singularly or in combination, with other pharmaceutical necessities included singularly or in combination, as desired, including:

tablet binders for compressed tablets, lozenges, and troches;

flavor oils such as peppermint, methyl salicylate, menthol and eucalyptol;

flavor oil stabilizers, including spray driers and cyclodextrins;

coloring agents and dyes;

glidants, including silica gel;

table lubricants, including magnesium stearate; and

other medicinal agents and nutritional supplements either directly incorporated within compositions or chemically isolated through techniques including microencapsulation and inclusion within cyclodextrins.

The compositions for use in the present invention include solid forms such as tablets, troches, lozenges, and powders; chewable forms such s chewing gums and soft candies; liquid forms such as syrups, mouth washes, and sprays. When these compositions are applied to the mouth of a human, they are palatable and without undesirable taste or unpleasant aftertaste.

Generally, lozenges are made in a 2 to 6 gram size to allow for a suitable dissolution rate for lozenges. Dissolution rate should be about 30 minutes when dissolved in the mouth as a lozenge, although there is considerable variability (fifteen minutes to one hour and fifteen minutes), depending upon the amount of saliva produced in response to the lozenges. As can be appreciated, the majority of the lozenges, perhaps up to 99 percent by weight, is pharmaceutical carrier.

Lozenges, tablets, and troches may differ in shape, size, and manufacturing technique. Hard candy lozenges made from sucrose and corn syrup or other melted hard candy bases can be used for incorporating the metal salts.

Soft delivery vehicles for the metal ions include soft candy, gum drop, liquid filled candies, or chewing gum base.

The metal ions can be administered in liquid compositions in a pharmaceutically acceptable liquid carrier. The metal ions can be administered in any liquid form such as syrups, mouth washes or sprays with water or other liquids for repeated delivery of ions to the oral mucous membranes over a sustained period of time so as to merit a prolonged contact of the ions in the mouth.

Sweeteners, such as fructose, sucrose, saccharide, acesulfame K, aspartame, cyclamates, monoammoniated glycyrrhizins, neoherperidin dihydrochalocone and other sweeteners can be added as needed to sweeten the compositions according to the present invention.

Any desired flavorings can be added to the compositions, such as anise, anethole, eucalyptol, wintergreen, licorice, clove, cinnamon, spearmint, cherry, lemon, orange, lime, menthol, peppermint, and combinations thereof. The delivery from a lozenge is significant in two (2) minutes, and onset of decongestion takes about the same time. The delivery from a lozenge is continual for as long as the supply of lozenge and buffering lasts. It is not, strictly speaking, necessary to have a dosage form held in the mouth. A nasal spray can also be used. The blockade lasts for several hours (between 2 to 4 hours). This can be extended by suitable carriers, ligands, and ligand-complexes.

For delivery of the metal ions from the mouth to the nose, the lozenge must be formulated to alter the pH of the mouth by buffering supplied by the lozenge to a pH of approximately 2.0 pH units or more below that of the nose. Thus, the combination of the metal salt and the buffering system must alter the pH of the mouth from an average of about 7.2 to about 4.8. As amino acids are preferred buffering agents, glycine is an appropriate choice. In this context, glycine serves both as an acid buffer and a complexing carrier agent.

A lozenge base for a metal salt according to the present invention can comprise about 50% sucrose, 83 grams fructose, 166 grams dextrose, 96 grams glycine, and 1.75 grams metal salt, e.g., zinc gluconate and aluminum gluconate. The amount of glycine as a buffer was determine to be a large molar excess to insure both pH and complexation of the metal ions chosen, and based on the average pH of the mouth to be 7.2. The steady state can be approximated by subtracting the first pKa of glycine from that of the mouth (7.2-2.34=4.86). In this example, the base contains much more glycine than metal salt. The buffering action will last as long as the lozenge is incompletely dissolved, and shortly thereafter, until all of the glycine is neutralized by the homeostatic mechanism of the mouth.

Alternatively, the metal ions can be delivered as a component of a breath strip or other patch type formulation. The metal ions can be delivered as sublingual liquids or pills, throat sprays, chewing gum, lollipops, gummy candies, and the like.

The foregoing description of the specific embodiments of the present invention will so fully reveal the general nature of the invention that others can, by applying current knowledge, readily modify and/or adapt for various application such specific embodiments without undue experimentation and without departing from the generic concept. Therefore, such adaptations and modifications should and are intended to be comprehended within the meaning and range of equivalents of the disclosed embodiments.

It is to be understood that the phraseology or terminology employed herein is for the purpose of description and not of limitation. The means and materials for carrying out disclosed functions may take a variety of alternative forms without departing form the invention. Thus, the expressions “means to . . .” and “means for . . .” as may be found the specification above, and/or in the claims below, followed by a functional statement, are intended to define and cover whatever structural, physical, chemical, or electrical element or structures which may now or in the future exist for carrying out the recited function, whether or not precisely equivalent to the embodiment or embodiments disclosed in the specification above, and it is intended that such expressions be given their broadest interpretation.

What is claimed is:

1. A method for inhibiting the calcium cascade comprising administering to an animal in need thereof an effective amount of a metal ion that blocks the calcium cascade.

2. The method according to claim 1 wherein the metal ions are selected from the group consisting of zinc, copper, magnesium, manganese, iron, and aluminum, and mixtures thereof.

3. The method according to claim 1 wherein the animal is suffering from an autoimmune disease which causes secretions and eruptions via the Calcium cascade.

4. The method according to claim 1 wherein the animal is suffering from rhinitis.

5. The method according to claim 1 wherein the animal is suffering from a herpes virus infection.

6. The method according to claim 1 wherein the metal ions are administered through the mouth to the nasal cavity.

7. The method according to claim 6 wherein the metal ions are in a composition which has a pH of about 4.8 so that the metal ions are delivered across the mucous membranes of the mouth into the nasal cavity.

8. The method according to claim 7 wherein the composition contains an amino acid as a buffer.

9. The method according to claim 8 wherein the amino acid is glycine.

10. The method according to claim 9 wherein the metal ion is zinc.

11. The method according to claim 10 wherein the metal ions are copper and zinc.
